(19) United States
(12) Patent Application Publication
(10) Pub. No.: US 2025/0204891 A1
(43) Pub. Date: Jun. 26, 2025

(54) CONTRAST ENHANCED ULTRASOUND METHOD, APPARATUS, COMPUTER DEVICE, AND STORAGE MEDIUM

(71) Applicant: United Imaging Research Institute of Intelligent Imaging, Beijing (CN)

(72) Inventors: Jian An, Beijing (CN); Fanglue Lin, Beijing (CN)

(21) Appl. No.: 18/991,834

(22) Filed: Dec. 23, 2024

(30) Foreign Application Priority Data
Dec. 22, 2023 (CN) 202311779693.4

(51) Int. Cl. A61B 8/00

(52) U.S. Cl. CPC A61B 8/5207; A61B 8/481; A61B 8/5238

(57) ABSTRACT

A CEUS method, an apparatus, a computer device, and a storage medium are provided. The method includes: acquiring channel data, adding the channel data with different attribute information, obtaining CEUS-complex data, performing beamforming on the channel data, obtaining complex channel data corresponding to the channel data, and obtaining a CEUS image according to the CEUS-complex data and the complex channel data.

### CROSS-REFERENCE TO RELATED APPLICATION

This application claims priority to Chinese patent application No. 202311779693.4, filed on Dec. 22, 2023, titled “CONTRAST ENHANCED ULTRASOUND METHOD, APPARATUS, COMPUTER DEVICE, AND STORAGE MEDIUM”, the content of which is hereby incorporated herein in its entirety by reference.

### TECHNICAL FIELD

The present disclosure generally relates to the field of medicine, and in particular, to a Contrast Enhanced Ultrasound (CEUS) method, an apparatus, a computer device, and a storage medium.

### BACKGROUND

CEUS is widely applied in the diagnosis of diseases. Currently, CEUS agents are injected intravenously to enhance blood flow signals of tissue organs and reflect blood perfusion better, thereby improving imaging quality of CEUS. Therefore, it is important to take into account a high signal-to-noise ratio and strong CEUS-tissue contrast to improve the imaging quality of CEUS.

### SUMMARY

According to various embodiments of the present disclosure, a CEUS method, an apparatus, a computer device, and a storage medium are provided.

In a first aspect, a CEUS method is provided in the present disclosure, including: acquiring channel data, adding the channel data with different attribute information, obtaining CEUS-complex data, performing beamforming on the channel data, obtaining complex channel data corresponding to the channel data, and obtaining a CEUS image according to the CEUS-complex data and the complex channel data.

In an embodiment, each of ultrasound pulse wave groups is corresponding to the channel data with different attribute information, and adding the channel data with different attribute information, and obtaining CEUS-complex data further includes: adding the channel data with different attribute information corresponding to each of ultrasound pulse wave groups, obtaining CEUS channel data, performing beamforming on the CEUS channel data, and obtaining the CEUS-complex data.

In an embodiment, the CEUS-complex data are arranged in a time sequence.

In an embodiment, after performing beamforming on the channel data, and obtaining complex channel data corresponding to the channel data, the method further includes: classifying the complex channel data according to the attribute information, and obtaining target complex data corresponding to the attribute information. Obtaining the CEUS image according to the CEUS-complex data and the complex channel data further includes: obtaining the CEUS image according to the CEUS-complex data and the target complex data corresponding to the attribute information.

In an embodiment, classifying the complex channel data according to the attribute information further includes: classifying the complex channel data with the same attribute information into a group. The target complex data includes the group of the complex channel data.

In an embodiment, before performing beamforming on the channel data, the method further includes: classifying the channel data with the same attribute information into a group. Performing beamforming on the channel data further includes: performing beamforming on the group of channel data.

In an embodiment, the target complex data corresponding to the attribute information are arranged in a time sequence.

In an embodiment, classifying the complex channel data according to the attribute information, and obtaining the target complex data corresponding to the attribute information further includes: classifying the complex channel data according to the attribute information, obtaining initial complex data corresponding to the attribute information, performing Doppler filtering on the initial complex data corresponding to the attribute information, and obtaining the target complex data corresponding to the attribute information.

In an embodiment, performing beamforming on the channel data, and obtaining complex channel data corresponding to the channel data further includes: performing beamforming on the channel data, performing Doppler filtering on the channel data after beamforming, and obtaining the complex channel data corresponding to the channel data.

In an embodiment, after obtaining the complex channel data corresponding to the channel data, the method further includes: classifying the complex channel data according to the attribute information, and obtaining target complex data corresponding to the attribute information. Obtaining the CEUS image according to the CEUS-complex data and the complex channel data further includes: obtaining the CEUS image according to the CEUS-complex data and the target complex data corresponding to the attribute information.

In an embodiment, the channel data is corresponding to N ultrasound pulse waves, N ultrasound pulse waves are divided into M ultrasound pulse wave groups, each of the M ultrasound pulse wave groups includes m ultrasound pulse waves, N, M, and m are positive integer greater than 1 and satisfy the following formula: M=N/m, and at least one of amplitude, phase, frequency, or distribution of activated elements are different in the m ultrasound pulse waves of each of the M ultrasound pulse wave groups.

In an embodiment, adding the channel data with different attribute information further includes: filtering out a non-linear fundamental component from the channel data with different attribute information.

In an embodiment, obtaining the CEUS image according to the CEUS-complex data and the target complex data corresponding to the attribute information further includes: generating a mask image according to the target complex data corresponding to the attribute information, performing updating on pixel values of the CEUS-complex data by the mask image, and obtaining the CEUS image.

In an embodiment, performing updating on pixel values of the CEUS-complex data by the mask image, and obtaining the CEUS image further includes: determining a first pixel in the mask image, a pixel value of the first pixel being a preset value, setting a pixel value of a second pixel in the CEUS-complex data to the preset value, and obtaining the CEUS image. The second pixel is corresponding to the first pixel.

In an embodiment, obtaining the CEUS image according to the CEUS-complex data and the target complex data corresponding to the attribute information further includes: for each third pixel in the CEUS-complex data, adding a pixel value of a fourth pixel in the target complex data corresponding to the attribute information and a pixel value of the third pixel, and obtaining the CEUS image. The third pixel is corresponding to the fourth pixel.

In an embodiment, the attribute information of the channel data includes at least one of amplitude, phase, frequency, or distribution of activated elements.

In an embodiment, the ultrasound pulse waves in each of the ultrasound pulse wave groups includes a wide beam, a plane wave, or a divergent wave.

In a second aspect, a CEUS apparatus is further provided in the present disclosure, including: means for acquiring channel data; means for adding the channel data with different attribute information, and obtaining CEUS-complex data; means for performing beamforming on the channel data, and obtaining complex channel data corresponding to the channel data; and means for obtaining a CEUS image according to the CEUS-complex data and the complex channel data.

In a third aspect, a computer device is further provided in the present disclosure, including a memory and a processor. A computer program is stored in the memory, and the processor is configured to execute the computer program to implement following steps: acquiring channel data, adding the channel data with different attribute information, obtaining CEUS-complex data, performing beamforming on the channel data, obtaining complex channel data corresponding to the channel data, and obtaining a CEUS image according to the CEUS-complex data and the complex channel data.

In a fourth aspect, a computer-readable storage medium is further provided in the present disclosure, which stores a computer program. The computer program is executed by a processor to implement following steps: acquiring channel data, adding the channel data with different attribute information, obtaining CEUS-complex data, performing beamforming on the channel data, obtaining complex channel data corresponding to the channel data, and obtaining a CEUS image according to the CEUS-complex data and the complex channel data.

In a fifth aspect, a computer program product is further provided in the present disclosure, which includes a computer program. The computer program is executed by a processor to implement following steps: acquiring channel data, adding the channel data with different attribute information, obtaining CEUS-complex data, performing beamforming on the channel data, obtaining complex channel data corresponding to the channel data, and obtaining a CEUS image according to the CEUS-complex data and the complex channel data.

Details of one or more embodiments of the present disclosure are set forth in the following accompanying drawings and description. Other features, objectives, and advantages of the present disclosure become obvious with reference to the specification, the accompanying drawings, and the claims.

### BRIEF DESCRIPTION OF THE DRAWINGS

In order to more clearly illustrate the technical solutions in the embodiments of the present disclosure or the related technology, the accompanying drawings to be used in the description of the embodiments or the related technology will be briefly introduced below, and it will be obvious that the accompanying drawings in the following description are only some of the embodiments of the present disclosure, and that, for one skilled in the art, other accompanying drawings can be obtained based on these accompanying drawings without putting in creative labor.

FIG. 1 is a schematic diagram of an application environment of a CEUS method in an embodiment.

FIG. 2A is a flowchart of a CEUS method in an embodiment.

FIG. 2B is a flowchart of a CEUS method in an embodiment.

FIG. 3 is a schematic diagram of a CEUS image in an embodiment.

FIG. 4 is a flowchart of a method of determining a target complex data in an embodiment.

FIG. 5 is a schematic diagram of a mask image in an embodiment.

FIG. 6 is a schematic diagram of a CEUS image in another embodiment.

FIG. 7 is a flowchart of a CEUS method in another embodiment.

FIG. 8 is a flowchart of a CEUS method in another embodiment.

FIG. 9 is a block diagram of a CEUS apparatus in an embodiment.

FIG. 10 is a schematic diagram of an internal structure of a computer device in an embodiment.

### DETAILED DESCRIPTION OF THE EMBODIMENT

To make objectives, technical solutions, and advantages of the present disclosure clearer, the following further describes the present disclosure in detail with reference to the accompanying drawings and embodiments. It should be understood that the specific embodiments described herein are merely used to explain the present disclosure, and are not intended to limit the present disclosure.

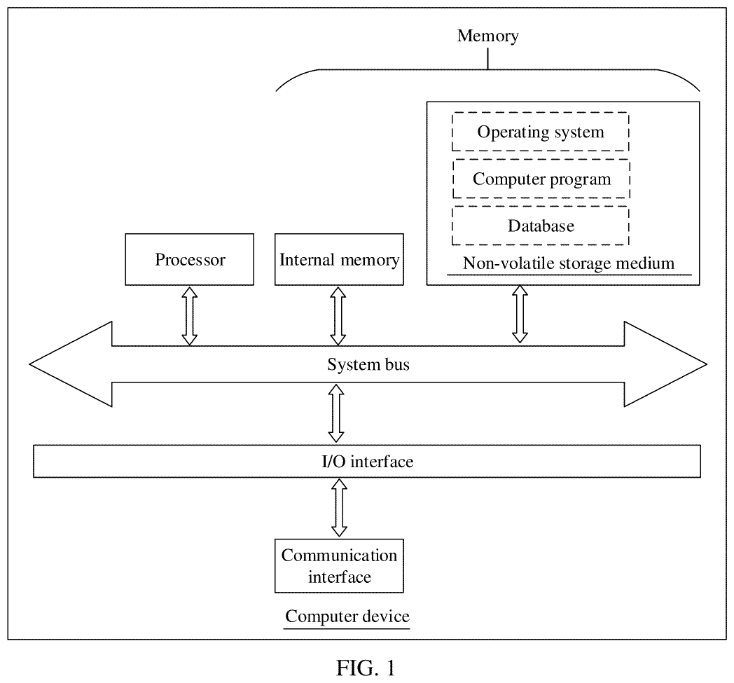

A CEUS method provided in an embodiment of the present disclosure may be applied to an application environment referring to FIG. 1. The application environment may include a computer device. The computer device may be a server, and a schematic diagram of an internal structure of the computer device may refer to FIG. 1. The computer device may include a processor, a memory, an Input/Output (I/O for short) interface, and a communication interface. The processor, the memory, and the I/O interface may be connected by a system bus, and the communication interface may be connected to the system bus by the I/O interface. The processor of the computer device is configured to provide a computing and control capability. The memory of the computer device may include a non-volatile storage medium and an internal memory. The non-volatile storage medium may store an operating system, a computer program, and a database. The internal storage may provide an environment for running the operating system and the computer program in the non-volatile storage medium. The database of the computer device is configured to store related data of CEUS. The I/O interface of the computer device is configured to exchange information between the processor and an external device. The communication interface of the computer device is configured to communicate with an external terminal by a network connection. The computer program is executed by a processor to implement the CEUS method. The server may be implemented by an independent server or a server cluster composed of multiple servers.

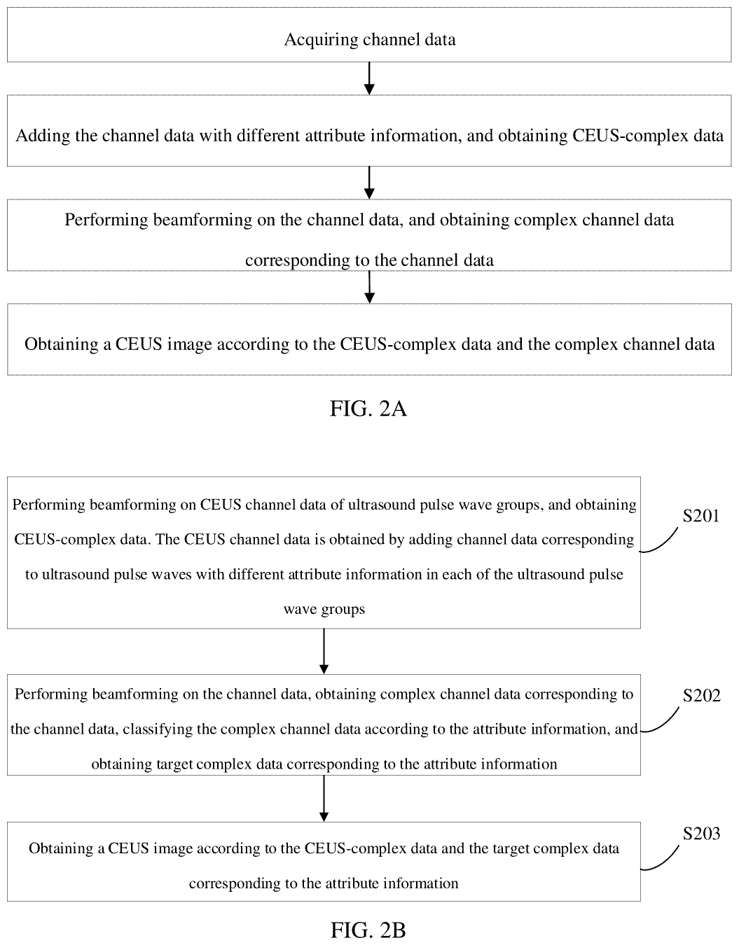

In an embodiment, referring to FIG. 2A, a CEUS method is provided, including: acquiring channel data, adding the channel data with different attribute information, obtaining CEUS-complex data, performing beamforming on the channel data, obtaining complex channel data corresponding to the channel data, and obtaining a CEUS image according to the CEUS-complex data and the complex channel data.

In an embodiment, each of ultrasound pulse wave groups is corresponding to the channel data with different attribute information, and adding the channel data with different attribute information, and obtaining CEUS-complex data may further include: adding the channel data with different attribute information corresponding to each of ultrasound pulse wave groups, obtaining CEUS channel data, performing beamforming on the CEUS channel data, and obtaining the CEUS-complex data.

In an embodiment, the CEUS-complex data may be arranged in a time sequence.

In an embodiment, after performing beamforming on the channel data, and obtaining complex channel data corresponding to the channel data, the method may further include: classifying the complex channel data according to the attribute information, and obtaining target complex data corresponding to the attribute information. Obtaining the CEUS image according to the CEUS-complex data and the complex channel data further includes: obtaining the CEUS image according to the CEUS-complex data and the target complex data corresponding to the attribute information.

In an embodiment, classifying the complex channel data according to the attribute information may further include: classifying the complex channel data with the same attribute information into a group. The target complex data may include the group of the complex channel data.

In an embodiment, before performing beamforming on the channel data, the method may further include: classifying the channel data with the same attribute information into a group. Performing beamforming on the channel data may further include: performing beamforming on the group of channel data.

In an embodiment, the target complex data corresponding to the attribute information may be arranged in a time sequence.

In an exemplary embodiment, referring to FIG. 2B, a CEUS method is provided for description by using an example in which the method is applied to the computer device in FIG. 1, and the method includes following step 201 and step 203.

Step 201 includes that performing beamforming on CEUS channel data of ultrasound pulse wave groups, and obtaining CEUS-complex data. The CEUS channel data is obtained by adding channel data corresponding to ultrasound pulse waves with different attribute information in each of the ultrasound pulse wave groups.

The channel data may be corresponding to ultrasound pulse waves, and the ultrasound waves may be divided into ultrasound pulse wave groups. The attribute information of the channel data may include at least one of amplitude, phase, frequency, or distribution of activated elements.

The attribute information of the ultrasound pulse waves in each of the ultrasound pulse wave groups may include at least one of amplitude, phase, frequency, or distribution of activated elements.

In the present disclosure, N ultrasound pulse waves may be transmitted, and a strong non-linear echo, i.e., corresponding N channel data, may be generated by a CEUS agent. The N ultrasound pulse waves may include M (M=N/m) ultrasound pulse wave groups, and at least one of amplitude, phase, frequency, or distribution of activated elements are different in m ultrasound pulse waves. For example, 80 ultrasound pulse waves may be sequentially transmitted in a time sequence, and a wave in front of an ultrasound pulse wave may be a plane wave. The ultrasound pulse waves may include two types of ultrasound pulse waves which are transmitted at intervals with a phase of 0° and a phase of 180° respectively. In this case, 40 ultrasound pulse wave groups may be included, and 80 pieces of the channel data may be obtained. Alternatively, 120 ultrasound pulse waves may be sequentially transmitted in a time sequence, and the wave in front of the ultrasound may be a plane wave. The 120 ultrasound pulse waves may be divided into 40 transmitting sets (i.e., 40 ultrasound pulse wave groups), and each of the transmitting sets may include three ultrasound pulse waves, which have a fixed sequence and may be denoted as A, B, and C, successively. A phase of A may be 0°, and activated elements may be all-channel enabled. A phase of B may be 180°, and all odd channels of the activated elements may be enabled. A phase of C may be 180°, and all even channels of the activated elements may be enabled.

Linear superposition may be performed on the m channel data in each of the ultrasound pulse groups, and after the linear superposition, M groups of CEUS channel data may be denoted as CE₁ to CE_{M}. The M groups of CEUS channel data CE₁ to CE_{M} may be arranged in a sequence of transmitting times of the ultrasound pulse waves. For example, channel data of the phase of 0° and the phase of 180° which are adjacent to each other in time may be superimposed to obtain 40 groups of CEUS channel data CE₁ to CE₄₀, and the 40 groups of CEUS channel data CE₁ to CE₄₀ may be arranged in a time sequence.

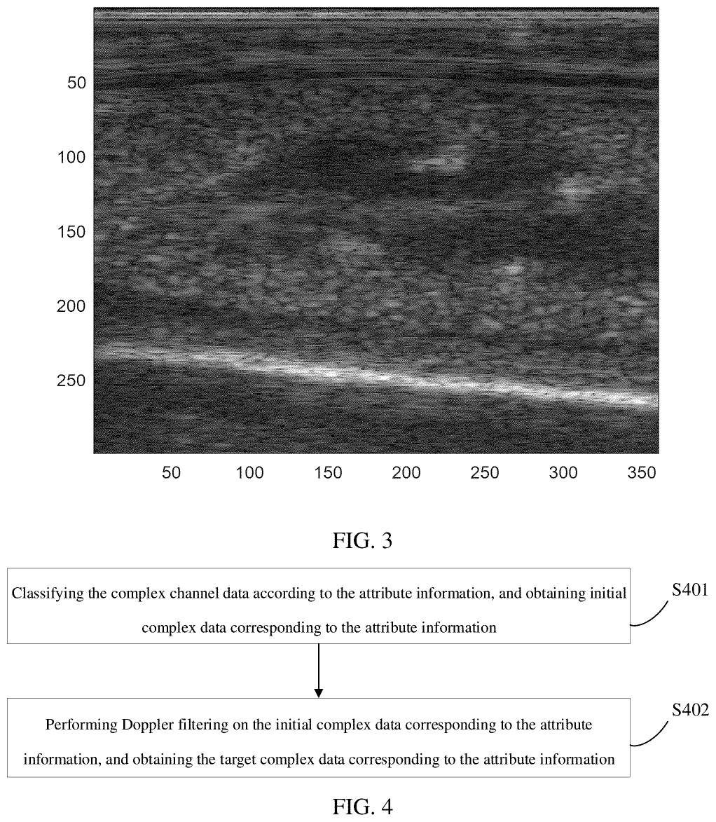

Beamforming may be performed on the M groups of CEUS channel data CE₁ to CE_{M}, respectively, to obtain CEUS-complex data, and the M groups of CEUS-complex data obtained after beamforming may be arranged in the time sequence. For example, beamforming may be performed on the 40 groups of CEUS channel data CE₁ to CE₄₀, respectively, to obtain CEUS-complex data denoted as ΣCEIQ after beamforming, ΣCEIQ may include 40 groups of CEUS-complex data CEIQ₁ to CEIQ₄₀ after beamforming, and the 40 groups of CEUS-complex data CEIQ_{I} to CEIQ₄₀ obtained after beamforming may be arranged in the time sequence. Referring to FIG. 3, it is a frame of a CEUS image generated based on CEIQ₁.

The channel data may be superimposed to extract a second harmonic component, a non-linear fundamental component, or the like. For example, the ultrasound pulse waves in the ultrasound pulse wave group are two ultrasound pulse waves between which transmitting phase difference is 180°, and the channel data may be superimposed to extract the second harmonic component. Multiple ultrasound pulse waves may be in the ultrasound pulse wave group with different amplitudes, and the channel data may be correspondingly superimposed to extract a non-linear fundamental component. The multiple ultrasound pulse waves may be in the ultrasound pulse wave group with different amplitudes and phase differences of 180°, and the channel data may be superimposed to extract a non-linear fundamental component, a second harmonic component, and the like, simultaneously.

Step 202 includes that performing beamforming on the channel data, obtaining complex channel data corresponding to the channel data, classifying the complex channel data according to the attribute information, and obtaining target complex data corresponding to the attribute information.

In the present embodiment, beamforming may be performed on the channel data to obtain the complex channel data corresponding to the channel data, and complex channel data with the same attribute information may be classified into one type according to the attribute information, to obtain the target complex data corresponding to the attribute information. In other words, beamforming may be performed on the channel data to obtain the complex channel data corresponding to the channel data, and complex channel data obtained after beamforming is performed on the channel data at a same transmitting position may be classified into one type, and the target complex data denoted as ΣDIQ₁ to ΣDIQ_{N/M} may be obtained after m(m=N/M) beams are beamformed. Each ΣDIQ may include target complex data denoted as DIQ₁ to DIQ_{M} obtained after beamforming of channel data of the same attribute information in M ultrasound pulse wave groups, and the target complex data DIQ₁ to DIQ_{M} may be arranged in the time sequence.

For example, the foregoing 80 ultrasound pulse waves may include channel data of ultrasound pulse waves with attribute information of the phase of 0° and the phase of 180°, beamforming may be performed on the channel data of 80 ultrasound pulse waves to obtain 80 pieces of corresponding plural data. Plural data corresponding to the phase of 0° may be classified into one type to obtain target complex data corresponding to the phase of 0°, and plural data corresponding to the phase of 180° may be classified into one type to obtain target complex data corresponding to the phase of 180°.

Alternatively, beamforming and classification may be performed on channel data of the foregoing 120 ultrasound pulse waves with three pieces of attribute information, i.e., plural data obtained after beamforming corresponding to the transmitted ultrasound pulse wave A may be classified into a first type, plural data obtained after beamforming corresponding to the transmitted ultrasound pulse wave B may be classified into a second type, and plural data obtained after beamforming corresponding to the transmitted ultrasound pulse wave C may be classified into a third type, so as to obtain three groups of target complex data denoted as ΣDIQ₁ to ΣDIQ₃ after beamforming. Each ΣDIQ may include target complex data DIQ₁ to DIQ₄₀ obtained after beamforming of 40 ultrasound pulse wave groups in the time sequence.

In an embodiment, after beamforming is performed on the channel data, Doppler filtering may be performed on the data after beamforming to obtain the complex channel data corresponding to the channel data. Furthermore, the complex channel data may be classified according to the attribute information to obtain the target complex data corresponding to the attribute information.

In another embodiment, after beamforming is performed on the channel data, the complex channel data may be obtained corresponding to the channel data, the complex channel data may be classified according to the attribute information to obtain initial complex data corresponding to the attribute information, and Doppler filtering may be performed on the initial complex data to obtain the target complex data corresponding to the attribute information.

Exemplarily, beamforming may be performed on 80 pieces of the channel data corresponding to the ultrasound pulse waves with the phase of 0° and the phase of 180° to obtain the complex channel data, and the complex channel data may be classified according to the attribute information to obtain initial complex data corresponding to the phase of 0° and initial complex data corresponding to the phase of 180°. Then, Doppler filtering may be performed on the initial complex data corresponding to the phase of 0° and the initial complex data corresponding to the phase of 180°, respectively, to obtain the target complex data corresponding to the phase of 0° and the phase of 180°.

Step 203 includes that obtaining a CEUS image according to the CEUS-complex data and the target complex data corresponding to the attribute information.

In the present embodiment, pixel operation may be performed on the CEUS-complex data ΣCEIQ and the target complex data ΣDIQ₁ to ΣDIQ_{N/M} corresponding to the attribute information after beamforming, to obtain the CEUS image. Specific pixel operation may include, but is not limited to, operation manners such as linear superposition, non-linear superposition, binarization mask, and cross-correlation. For example, taking the foregoing 80 pulse wave data as an example, pixel operation may be performed on CEIQ, DIQ₁, DIQ₂, and DIQ₃ corresponding to the time sequence, to obtain a final CEUS image ΣCEUS. The ΣCEUS may include 40 CEUS images denoted as CEUS₁ to CEUS₄₀.

In the foregoing CEUS method, beamforming is performed on the CEUS channel data of the ultrasound pulse wave groups to obtain the CEUS-complex data. Beamforming is performed on the channel data to obtain complex channel data corresponding to the channel data, the complex channel data is classified according to the attribute information to obtain target complex data corresponding to the attribute information, and the ultrasound imaging image is obtained according to the CEUS-complex data and the target complex data corresponding to the attribute information. The CEUS channel data is obtained by adding channel data corresponding to ultrasound pulse waves with different attribute information in the ultrasound pulse wave groups. In the present embodiment of the present disclosure, the target complex data corresponding to the attribute information and the CEUS-complex data may be beamformed, so as to enhance CEUS-to-tissue contrast and improve a signal-to-noise ratio of the CEUS image. The channel data corresponding to ultrasound pulse wave with different attribute information may be superimposed, so as to filter out a fundamental component signal from the channel data and retain a harmonic signal, so that tissue echo may be suppressed, thereby resolving a problem that it is difficult to identify the CEUS agent with a relatively slow moving speed.

FIG. 4 is a flowchart of a method of determining the target complex data in an embodiment. Referring to FIG. 4, an embodiment of the present disclosure relates to a possible implementation of how to classify the complex channel data according to the attribute information and obtain the target complex data corresponding to the attribute information, including following step 401 and step 402.

Step 401 may include that classifying the complex channel data according to the attribute information, and obtaining initial complex data corresponding to the attribute information.

Step 402 may include that performing Doppler filtering on the initial complex data corresponding to the attribute information, and obtaining the target complex data corresponding to the attribute information.

Doppler filtering may be at least one of a finite impulse response filtering algorithm, an infinite impulse response filtering algorithm, a polynomial filtering algorithm, an eigenvalue decomposition algorithm, a low rank matrix decomposition algorithm, a robust principal component analysis algorithm, a singular value decomposition algorithm, or an independent component analysis algorithm.

In the present embodiment, complex channel data with the same attribute information may be classified into one type according to the attribute information, and the initial complex data denoted as ΣIQ₁ to ΣIQ_{N/M} corresponding to the attribute information may be obtained. The initial complex data ΣIQ₁ to ΣIQ_{N/M} may be arranged in the time sequence. Doppler filtering may be performed on the initial complex data ΣIQ₁-ΣIQ_{N/M} to obtain the target complex data ΣDIQ₁ to ΣDIQ_{N/M} corresponding to the attribute information.

In the present embodiment of the present disclosure, the complex channel data may be classified according to the attribute information to obtain the initial complex data corresponding to the attribute information, and Doppler filtering may be performed on the initial complex data corresponding to the attribute information to obtain the target complex data corresponding to the attribute information. In the present embodiment of the present disclosure, Doppler filtering may be used to obtain the target complex data, which may resolve a problem of incomplete tissue suppression, and CEUS agent duration may be maintained better, thereby improving imaging quality of the CEUS image determined based on the target complex data and the CEUS-complex data.

In an embodiment, it relates to two possible implementations of how to obtain the CEUS image according to the CEUS-complex data and the target complex data corresponding to the attribute information.

A first implementation may include that generating a mask image according to the target complex data corresponding to the attribute information, performing updating on pixel values of the CEUS-complex data by the mask image, and obtaining the CEUS image.

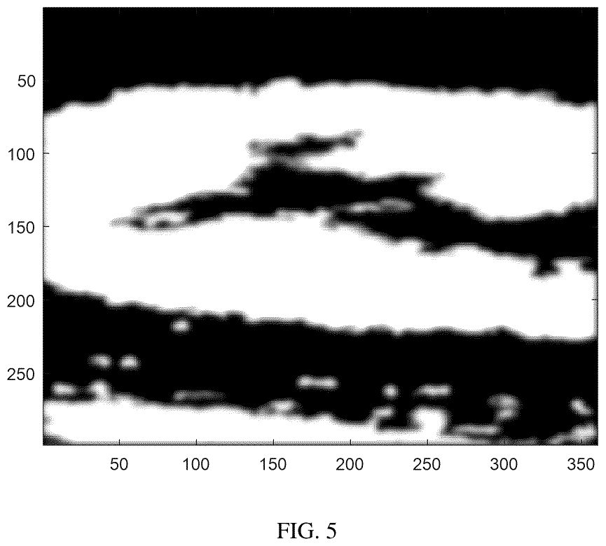

In the present embodiment, the target complex data corresponding to the same attribute information may be superimposed to obtain a superimposed image, the superimposed image may be converted into a corresponding binary image according to a preset threshold, and an intersection set of binary images corresponding to the attribute information may be taken as a final mask image. The foregoing 80 ultrasound pulse waves may be taken as an example. An ultrasonic pulse wave with the phase of 0° may corresponding to target complex data IQ₁-IQ₄₀, and an ultrasonic pulse wave with the phase of 180° may corresponding to target complex data IQ₁-IQ₄₀. The target complex data IQ₁-IQ₄₀ corresponding to the phase of 0° may be superimposed to obtain a superimposed image 1, and the target complex data IQ₁-IQ₄₀ corresponding to the phase of 180° may be superimposed to obtain a superimposed image 2. The threshold may be set to 50, a pixel value in the superimposed image 1 less than 50 may be set to 0, and a pixel value in the superimposed image 1 greater than or equal to 50 may be set to 1, so as to obtain a binary image 1. Similarly, for the superimposed image 2, a pixel value in the superimposed image 2 less than 50 may be set to 0, and a pixel value in the superimposed image 2 greater than or equal to 50 may be set to 2, so as to obtain a binary image 2. A pixel value of a pixel of the binary image 1 may be compared with that of a corresponding pixel of the binary image 2, if pixel values of the corresponding pixel are consistent with each other, the pixel value of the corresponding pixel may be set to 1, otherwise, the pixel value of the corresponding pixel may be set to 0, so as to obtain the mask image referring to FIG. 5.

A first pixel in the mask image may be determined first, a pixel value of the first pixel is a preset value, a pixel value of a second pixel in the CEUS-complex data may be set to a preset value to obtain the CEUS image, and the second pixel is corresponding to the first pixel. Alternatively, weighted processing may be performed on the pixel value of the second pixel in the CEUS-complex data to obtain a new pixel value, so as to obtain the CEUS image.

A second implementation may include that for each third pixel in the CEUS-complex data, adding a pixel value of a fourth pixel in the target complex data corresponding to the attribute information and a pixel value of the third pixel, and obtaining the CEUS image. The third pixel is corresponding to the fourth pixel.

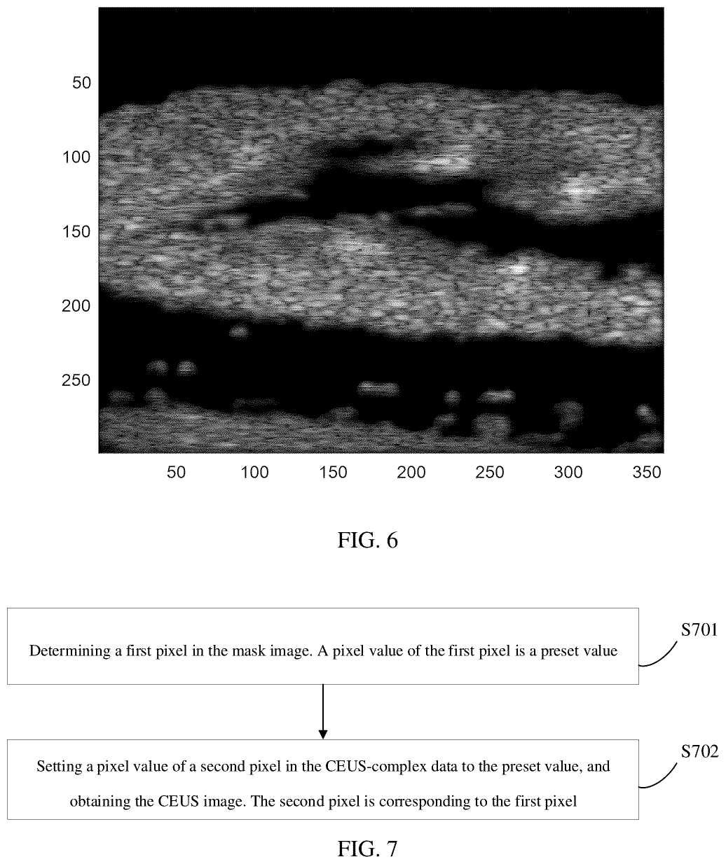

In the present embodiment of the present disclosure, since the CEUS-complex data ΣCEIQ and the target complex data ΣDIQ₁ to ΣDIQ_{N/M} corresponding to the attribute information are arranged in the time sequence, for each third pixel in the CEUS-complex data, the pixel value of the fourth pixel in the target complex data corresponding to the attribute information and the pixel value of the third pixel may be superimposed to obtain the CEUS image. For example, a pixel value of the corresponding pixel in the CEUS-complex data CEIQ₁, a pixel value of the corresponding pixel in the target complex data DIQ₁ with the phase of 0°, and a pixel value of the corresponding pixel in the target complex data DIQ₁ with the phase of 180° may be superimposed to obtain the CEUS image CEUS₁ referring to FIG. 6.

In the present embodiment of the present disclosure, the CEUS image may be obtained according to the CEUS-complex data and the target complex data corresponding to the attribute information. The CEUS-complex data and the target complex data corresponding to the attribute information may be synthetized, which may resolve problems that a signal-to-noise ratio of the channel data is relatively low, a penetration of the CEUS image is relatively poor, and it is difficult to identify the CEUS agent with a relatively slow moving speed, resulting in missing a final CEUS signal. Furthermore, the CEUS-tissue contrast may be enhanced, and imaging quality of the CEUS image may be improved.

FIG. 7 is a flowchart of a CEUS method in another embodiment. Referring to FIG. 7, an embodiment of the present disclosure relates to a possible implementation of how to perform updating on pixel values of the CEUS-complex data by the mask image and obtain the CEUS image, including following step 701 and step 702.

Step 701 may include that determining a first pixel in the mask image, and a pixel value of the first pixel is a preset value.

Step 702 may include that setting a pixel value of a second pixel in the CEUS-complex data to the preset value, and obtaining the CEUS image. The second pixel is corresponding to the first pixel.

Alternatively, the preset value may be 0. The pixel value of the second pixel in the CEUS-complex data may be set to the preset value to obtain the CEUS image, i.e., the mask image may be used to perform a zeroing operation on the CEUS-complex data ΣCEIQ. In other words, 40 CEUS images CEIQ₁ to CEIQ₄₀ may be compared with the mask image, respectively, a pixel of pixel value 1 in the mask image may keep an original pixel value on the CEUS-complex data, and a pixel of pixel value 0 in the mask image may be set to 0 on the CEUS-complex data, so as to finally obtain the CEUS image ΣCEUS. ΣCEUS may include 40 CEUS images CEUS₁ to CEUS₄₀.

In the present embodiment of the present disclosure, the first pixel may be determined in the mask image, the pixel value of the first pixel is the preset value, and the pixel value of the second pixel in the CEUS-complex data corresponding to the first pixel may be set to the preset value, so as to obtain the CEUS image. In the present embodiment, the mask image may be determined according to the target complex data corresponding to the attribute information, and the mask image may be used to update the CEUS-complex data to obtain the CEUS image, so as to combine the target complex data and the CEUS-complex data effectively, and avoid tissue residue on the generated CEUS image, i.e., the CEUS-tissue contrast of the CEUS image is relatively poor. Furthermore, it may be avoided that a frame frequency difference of CEUS due to vulnerability of CEUS agents, and artificially reducing a repetition rate of ultrasound pulse wave emission to maintain sufficient CEUS agent duration.

In an embodiment, the ultrasound pulse waves in each of the ultrasound pulse wave groups may include wide beams or plane wave and divergent wave, which may ensure that the final CEUS image has an advantage of a high imaging frame frequency.

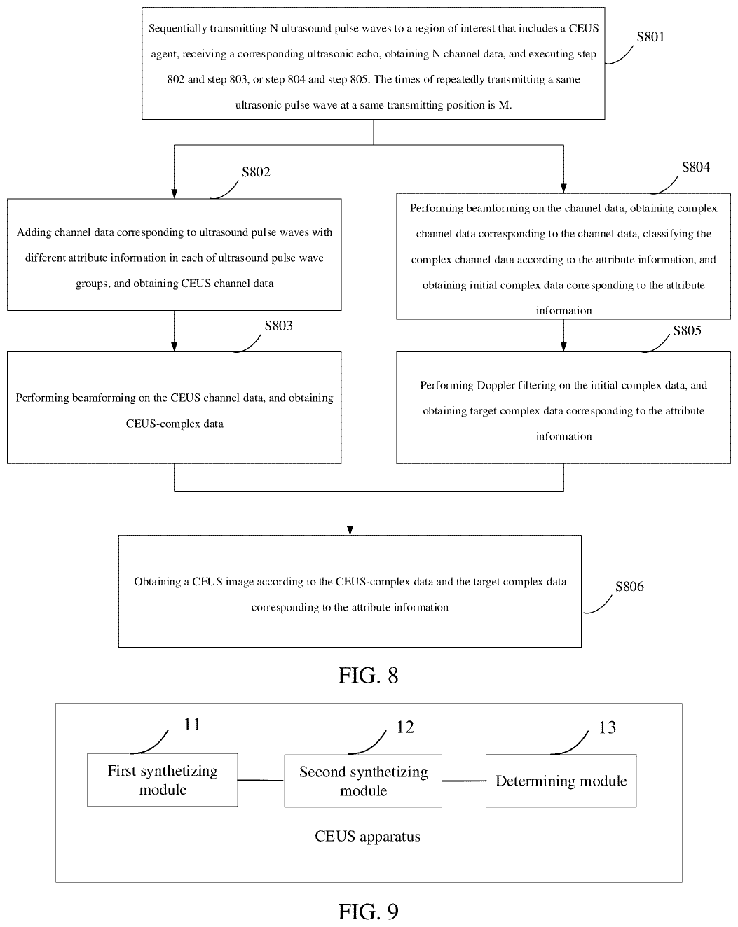

FIG. 8 is a flowchart of a CEUS method in another embodiment. Referring to FIG. 8, the CEUS method may include following step 801 to step 806.

Step 801 may include that sequentially transmitting N ultrasound pulse waves to a region of interest that includes a CEUS agent, receiving a corresponding ultrasonic echo, obtaining N channel data, and executing step 802 and step 803, or step 804 and step 805. The times of repeatedly transmitting a same ultrasonic pulse wave at a same transmitting position is M.

Step 802 may include that adding channel data corresponding to ultrasound pulse waves with different attribute information in each of ultrasound pulse wave groups, and obtaining CEUS channel data.

Step 803 may include that performing beamforming on the CEUS channel data, and obtaining CEUS-complex data.

Step 804 may include that performing beamforming on the channel data, obtaining complex channel data corresponding to the channel data, classifying the complex channel data according to the attribute information, and obtaining initial complex data corresponding to the attribute information.

Step 805 may include that performing Doppler filtering on the initial complex data, and obtaining target complex data corresponding to the attribute information.

Step 806 may include that obtaining a CEUS image according to the CEUS-complex data and the target complex data corresponding to the attribute information.

In the present embodiment of the present disclosure, the target complex data corresponding to the attribute information and the CEUS-complex data may be synthetized, so as to enhance the CEUS-tissue contrast and improve the signal-to-noise ratio of the CEUS image. The channel data corresponding to ultrasound pulse waves with different attribute information may be superimposed, so as to filter the fundamental component signal in the CEUS channel data and retain the harmonic signal, so that tissue echo may be suppressed, thereby resolving the problem that it is difficult to identify the CEUS agent with the relatively slow moving speed.

It should be understood that, although steps in the flowchart related to the foregoing embodiments are sequentially displayed according to an instruction of an arrow, these steps are not necessarily sequentially performed according to the instruction of the arrow. Unless expressly stated in this specification, these steps are not performed in a strict order, and these steps may be performed in another order. In addition, at least a part of steps in the flowchart involved in the foregoing embodiments may include multiple steps or multiple phases. These steps or phases are not necessarily performed at a same moment, but may be performed at different moments. These steps or phases are not necessarily performed sequentially, but may be performed alternately or alternately with another step or at least a part of steps or phases in another step.

Based on the same invention concept, an embodiment of the present disclosure may further provide a CEUS apparatus configured to implement the foregoing involved CEUS method. An implementation solution provided by the apparatus is similar to the implementation solution described in the foregoing method. Therefore, a specific limitation in one or more embodiments of the CEUS apparatus provided below may refer to the foregoing limitation in the CEUS method. Details are not described herein again.

In an exemplary embodiment, referring to FIG. 9, a CEUS apparatus is provided, including a first synthetizing module 11, a second synthetizing module 12, and a determining module 13.

The first synthetizing module 11 is configured for performing beamforming on CEUS channel data of ultrasound pulse wave groups, and obtaining CEUS-complex data. The CEUS channel data is obtained by adding channel data corresponding to ultrasound pulse waves with different attribute information in each of the ultrasound pulse wave groups.

The second synthetizing module 12 is configured for performing beamforming on the channel data, obtaining plural data corresponding to the channel data, classifying the complex channel data according to the attribute information, and obtaining target complex data corresponding to the attribute information.

The determining module 13 is configured for obtaining a CEUS image according to the CEUS-complex data and the target complex data corresponding to the attribute information.

In an embodiment, the second synthetizing module 12 may further include a classifying unit and a processing unit. The classifying unit is configured for classifying the complex channel data according to the attribute information, and obtaining initial complex data corresponding to the attribute information. The processing unit is configured for performing Doppler filtering on the initial complex data corresponding to the attribute information, and obtaining the target complex data corresponding to the attribute information.

In an embodiment, the determining module 13 may further include a generating unit and an updating unit. The generating unit is configured for generating a mask image according to the target complex data corresponding to the attribute information. The updating unit is configured for performing updating on pixel values of the CEUS-complex data by the mask image, and obtaining the CEUS image.

In an embodiment, the updating unit is further configured for determining a first pixel in the mask image, setting a pixel value of a second pixel in the CEUS-complex data to a preset value, and obtaining the CEUS image. A pixel value of the first pixel is the preset value, and the second pixel is corresponding to the first pixel.

In an embodiment, the determining module 13 may further include a adding unit. For each third pixel in the CEUS-complex data, the adding unit is configured for adding a pixel value of a fourth pixel in the target complex data corresponding to the attribute information and a pixel value of the third pixel, and obtaining the CEUS image. The third pixel is corresponding to the fourth pixel.

In an embodiment, the attribute information of the ultrasound pulse waves in each of the ultrasound pulse wave groups may include at least one of amplitude, phase, frequency, or distribution of activated elements.

In an embodiment, the ultrasound pulse waves in each of the ultrasound pulse wave groups may include wide beams or plane wave and divergent wave.

It should be noted that all modules in the foregoing CEUS apparatus may be implemented in whole or in part by software, hardware, and a combination thereof. The foregoing modules may be embedded in or independent of a processor in a computer device in a hardware form, or may be stored in a memory in the computer device in a software form, so that the processor may invoke to execute an operation corresponding to the foregoing modules.

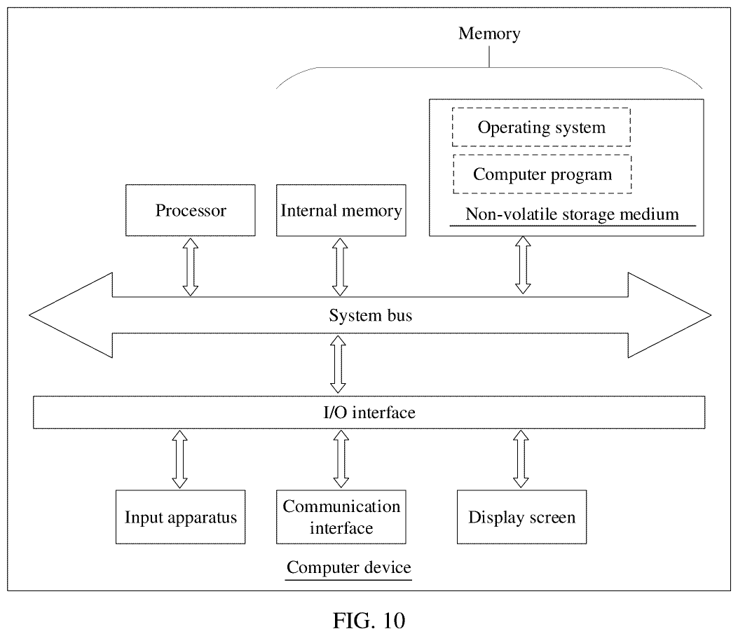

In an exemplary embodiment, a computer device is further provided, the computer device may be a terminal, and an internal structure diagram of the computer device may refer to FIG. 10. The computer device may include a processor, a memory, an input/output interface, a communication interface, a display unit, and an input apparatus. The processor, the memory, and the input/output interface may be connected by the system bus, and the communication interface, the display unit, and the input apparatus may be connected to the system bus by the input/output interface. The processor of the computer device is configured to provide a computing and control capability. The memory of the computer device may include a non-volatile storage medium and an internal memory. The non-volatile storage medium may store an operating system and a computer program. The internal storage may provide an environment for running the operating system and the computer program in the non-volatile storage medium. The input/output interface of the computer device is configured to exchange information between the processor and an external device. The communication interface of the computer device is configured to communicate with an external terminal in a wired or wireless manner. The wireless manner may be implemented by a WIFI, a mobile cellular network, an NFC (near field communication), or other technology. The computer program is executed by the processor to implement the CEUS method. The display unit of the computer device is configured to form a visual picture, which may be a display screen, a projection apparatus, or a virtual reality imaging apparatus. The display screen may be a liquid crystal display screen or an electronic ink display screen. The input apparatus of the computer device may be a touch layer covered on the display screen, may be a key, a trackball, or a touchpad disposed on a housing of the computer device, or may be an external keyboard, a touchpad, a mouse, or the like.

One skilled in the art may understand that the structure shown in FIG. 10 is merely a block diagram of some structures related to the solutions of the present disclosure, and does not constitute a limitation on the computer device to which the solutions of the present disclosure are applied. A specific computer device may include more or fewer members than those shown in the figure, or combine some members, or have different member arrangements.

In an exemplary embodiment, a computer device is further provided, including a memory and a processor. A computer program is stored in the memory, and the processor is configured to execute the computer program to implement following steps: acquiring channel data, adding the channel data with different attribute information, obtaining CEUS-complex data, performing beamforming on the channel data, obtaining complex channel data corresponding to the channel data, and obtaining a CEUS image according to the CEUS-complex data and the complex channel data.

In an embodiment, each of ultrasound pulse wave groups is corresponding to the channel data with different attribute information, and the processor is configured to execute the computer program to implement following steps: adding the channel data with different attribute information, obtaining CEUS channel data corresponding to each of ultrasound pulse wave groups, performing beamforming on the CEUS channel data, and obtaining the CEUS-complex data.

In an embodiment, the CEUS-complex data of the ultrasound pulse wave groups are arranged in a time sequence.

In an embodiment, the processor is configured to execute the computer program to implement following steps: after performing beamforming on the channel data, and obtaining complex channel data corresponding to the channel data, classifying the complex channel data according to the attribute information, and obtaining target complex data corresponding to the attribute information. Obtaining the CEUS image according to the CEUS-complex data and the complex channel data further includes: obtaining the CEUS image according to the CEUS-complex data and the target complex data corresponding to the attribute information.

In an embodiment, the processor is configured to execute the computer program to implement following steps: classifying the complex channel data with the same attribute information into a group. The target complex data may include the group of the complex channel data.

In an embodiment, the processor is configured to execute the computer program to implement following steps: before performing beamforming on the channel data, classifying the channel data with the same attribute information into a group, and performing beamforming on the group of channel data.

In an embodiment, the target complex data corresponding to the attribute information are arranged in a time sequence.

In an embodiment, the processor is configured to execute the computer program to implement following steps: classifying the complex channel data according to the attribute information, obtaining initial complex data corresponding to the attribute information, performing

Doppler filtering on the initial complex data corresponding to the attribute information, and obtaining the target complex data corresponding to the attribute information.

In an embodiment, the processor is configured to execute the computer program to implement following steps: performing beamforming on the channel data, performing Doppler filtering on the channel data after beamforming, and obtaining the complex channel data corresponding to the channel data.

In an embodiment, the processor is configured to execute the computer program to implement following steps: after obtaining the complex channel data corresponding to the channel data, classifying the complex channel data according to the attribute information, obtaining target complex data corresponding to the attribute information, and obtaining the CEUS image according to the CEUS-complex data and the target complex data corresponding to the attribute information.

In an embodiment, the processor is configured to execute the computer program to implement following steps: filtering out a non-linear fundamental component from the channel data with different attribute information.

In an embodiment, the processor is configured to execute the computer program to implement following steps: generating a mask image according to the target complex data corresponding to the attribute information, performing updating on pixel values of the CEUS-complex data by the mask image, and obtaining the CEUS image.

In an embodiment, the processor is configured to execute the computer program to implement following steps: determining a first pixel in the mask image, a pixel value of the first pixel being a preset value, setting a pixel value of a second pixel in the CEUS-complex data to the preset value, and obtaining the CEUS image. The second pixel is corresponding to the first pixel.

In an embodiment, the processor is configured to execute the computer program to implement following steps: for each third pixel in the CEUS-complex data, adding a pixel value of a fourth pixel in the target complex data corresponding to the attribute information and a pixel value of the third pixel, and obtaining the CEUS image. The third pixel is corresponding to the fourth pixel.

In an embodiment, the attribute information of the channel data may include at least one of amplitude, phase, frequency, or distribution of activated elements.

In an embodiment, the ultrasound pulse waves in each of the ultrasound pulse wave groups may include wide beams or plane wave and divergent wave.

In an embodiment, a computer-readable storage medium is further provided, which stores a computer program. The computer program is executed by a processor to implement following steps: acquiring channel data, adding the channel data with different attribute information, obtaining CEUS-complex data, performing beamforming on the channel data, obtaining complex channel data corresponding to the channel data, and obtaining a CEUS image according to the CEUS-complex data and the complex channel data.

In an embodiment, each of ultrasound pulse wave groups is corresponding to the channel data with different attribute information, and the computer program is executed by the processor to implement following steps: adding the channel data with different attribute information corresponding to each of ultrasound pulse wave groups, obtaining CEUS channel data, performing beamforming on the CEUS channel data, and obtaining the CEUS-complex data.

In an embodiment, the CEUS-complex data of the ultrasound pulse wave groups are arranged in a time sequence.

In an embodiment, the computer program is executed by the processor to implement following steps: after performing beamforming on the channel data, and obtaining complex channel data corresponding to the channel data, classifying the complex channel data according to the attribute information, and obtaining target complex data corresponding to the attribute information. Obtaining the CEUS image according to the CEUS-complex data and the complex channel data further includes: obtaining the CEUS image according to the CEUS-complex data and the target complex data corresponding to the attribute information.

In an embodiment, the computer program is executed by the processor to implement following steps: classifying the complex channel data with the same attribute information into a group. The target complex data may include the group of the complex channel data.

In an embodiment, the computer program is executed by the processor to implement following steps: before performing beamforming on the channel data, classifying the channel data with the same attribute information into a group, and performing beamforming on the group of channel data.

In an embodiment, the target complex data corresponding to the attribute information are arranged in a time sequence.

In an embodiment, the computer program is executed by the processor to implement following steps: classifying the complex channel data according to the attribute information, obtaining initial complex data corresponding to the attribute information, performing Doppler filtering on the initial complex data corresponding to the attribute information, and obtaining the target complex data corresponding to the attribute information.

In an embodiment, the computer program is executed by the processor to implement following steps: performing beamforming on the channel data, performing Doppler filtering on the channel data after beamforming, and obtaining the complex channel data corresponding to the channel data.

In an embodiment, the computer program is executed by the processor to implement following steps: after obtaining the complex channel data corresponding to the channel data, classifying the complex channel data according to the attribute information, obtaining target complex data corresponding to the attribute information, and obtaining the CEUS image according to the CEUS-complex data and the target complex data corresponding to the attribute information.

In an embodiment, the computer program is executed by the processor to implement following steps: filtering out a non-linear fundamental component from the channel data with different attribute information.

In an embodiment, the computer program is executed by the processor to implement following steps: generating a mask image according to the target complex data corresponding to the attribute information, performing updating on pixel values of the CEUS-complex data by the mask image, and obtaining the CEUS image.

In an embodiment, the computer program is executed by the processor to implement following steps: determining a first pixel in the mask image, a pixel value of the first pixel being a preset value, setting a pixel value of a second pixel in the CEUS-complex data to the preset value, and obtaining the CEUS image. The second pixel is corresponding to the first pixel.

In an embodiment, the computer program is executed by the processor to implement following steps: for each third pixel in the CEUS-complex data, adding a pixel value of a fourth pixel in the target complex data corresponding to the attribute information and a pixel value of the third pixel, and obtaining the CEUS image. The third pixel is corresponding to the fourth pixel.

In an embodiment, the attribute information of the channel data may include at least one of amplitude, phase, frequency, or distribution of activated elements.

In an embodiment, the ultrasound pulse waves in each of the ultrasound pulse wave groups may include wide beams or plane wave and divergent wave.

In an embodiment, a computer program product is further provided in the present disclosure, which includes a computer program. The computer program is executed by a processor to implement following steps: acquiring channel data, adding the channel data with different attribute information, obtaining CEUS-complex data, performing beamforming on the channel data, obtaining complex channel data corresponding to the channel data, and obtaining a CEUS image according to the CEUS-complex data and the complex channel data.

In an embodiment, each of ultrasound pulse wave groups is corresponding to the channel data with different attribute information, and the computer program is executed by the processor to implement following steps: adding the channel data with different attribute information corresponding to each of ultrasound pulse wave groups, obtaining CEUS channel data, performing beamforming on the CEUS channel data, and obtaining the CEUS-complex data.

In an embodiment, the CEUS-complex data of the ultrasound pulse wave groups are arranged in a time sequence.

In an embodiment, the computer program is executed by the processor to implement following steps: after performing beamforming on the channel data, and obtaining complex channel data corresponding to the channel data, classifying the complex channel data according to the attribute information, and obtaining target complex data corresponding to the attribute information. Obtaining the CEUS image according to the CEUS-complex data and the complex channel data further includes: obtaining the CEUS image according to the CEUS-complex data and the target complex data corresponding to the attribute information.

In an embodiment, the computer program is executed by the processor to implement following steps: classifying the complex channel data with the same attribute information into a group. The target complex data may include the group of the complex channel data.

In an embodiment, the computer program is executed by the processor to implement following steps: before performing beamforming on the channel data, classifying the channel data with the same attribute information into a group, and performing beamforming on the group of channel data.

In an embodiment, the target complex data corresponding to the attribute information are arranged in a time sequence.

In an embodiment, the computer program is executed by the processor to implement following steps: classifying the complex channel data according to the attribute information, obtaining initial complex data corresponding to the attribute information, performing Doppler filtering on the initial complex data corresponding to the attribute information, and obtaining the target complex data corresponding to the attribute information.

In an embodiment, the computer program is executed by the processor to implement following steps: performing beamforming on the channel data, performing Doppler filtering on the channel data after beamforming, and obtaining the complex channel data corresponding to the channel data.

In an embodiment, the computer program is executed by the processor to implement following steps: after obtaining the complex channel data corresponding to the channel data, classifying the complex channel data according to the attribute information, obtaining target complex data corresponding to the attribute information, and obtaining the CEUS image according to the CEUS-complex data and the target complex data corresponding to the attribute information.

In an embodiment, the computer program is executed by the processor to implement following steps: filtering out a non-linear fundamental component from the channel data with different attribute information.

In an embodiment, the computer program is executed by the processor to implement following steps: generating a mask image according to the target complex data corresponding to the attribute information, performing updating on pixel values of the CEUS-complex data by the mask image, and obtaining the CEUS image.

In an embodiment, the computer program is executed by the processor to implement following steps: determining a first pixel in the mask image, a pixel value of the first pixel being a preset value, setting a pixel value of a second pixel in the CEUS-complex data to the preset value, and obtaining the CEUS image. The second pixel is corresponding to the first pixel.

In an embodiment, the computer program is executed by the processor to implement following steps: for each third pixel in the CEUS-complex data, adding a pixel value of a fourth pixel in the target complex data corresponding to the attribute information and a pixel value of the third pixel, and obtaining the CEUS image. The third pixel is corresponding to the fourth pixel.

In an embodiment, the attribute information of the channel data may include at least one of amplitude, phase, frequency, or distribution of activated elements.

In an embodiment, the ultrasound pulse waves in each of the ultrasound pulse wave groups may include wide beams or plane wave and divergent wave.

It should be noted that user information (including but not limited to user device information, user personal information, and the like) and data (including but not limited to data used for analysis, stored data, and displayed data) involved in the present disclosure are information and data that are authorized by the user or that are fully authorized by each party, and collection, use, and processing of related data need to comply with related regulations.

One skilled in the art may understand that all or a part of the processes in the methods in the foregoing embodiments may be implemented by a computer program instructing related hardware. The computer program may be stored in a non-volatile computer readable storage medium. When the computer program is executed, the processes in the foregoing methods embodiments may be included. Any reference to a memory, a database, or other medium used in the embodiments provided in the present disclosure may include at least one of a non-volatile memory or a volatile memory. The non-volatile memory may include a Read-Only Memory (ROM), a magnetic tape, a floppy disk, a flash memory, an optical memory, a high-density embedded non-volatile memory, a Resistive Random Access Memory (ReRAM), a Magneto resistive Random Access Memory (MRAM), a Ferroelectric Random Access Memory (FRAM), a Phase Change Memory (PCM), a graphene memory, and the like. The volatile memory may include a Random Access Memory (RAM), an external cache, or the like. As an illustration and not a limitation, the RAM may be in multiple forms, such as a Static Random Access Memory (SRAM) or a Dynamic Random Access Memory (DRAM). The databases involved in the embodiments provided in the present disclosure may include at least one of a relational database or a non-relational database. The non-relational database may include a distributed database based on a block chain or the like, which is not limited thereto. The processor in the embodiments provided in present disclosure may be a general-purpose processor, a central processing unit, a graphics processor, a digital signal processor, a programmable logic device, a quantum computing-based data processing logic device, or the like, which is not limited thereto.

All the technical features in the foregoing embodiments may be any combination. To make the description brief, all possible combinations of the technical features in the foregoing embodiments are not described. However, as long as there is no contradiction between the combinations of the technical features, it should be considered as the scope described in this specification.

The foregoing embodiments represent only several implementation manners of the present disclosure, and descriptions thereof are relatively specific and detailed, but may not be construed as a limitation on the scope of the present disclosure. It should be noted that one skilled in the art may make some modifications and improvements without departing from the concept of the present disclosure, which are within the protection scope of the present disclosure. Therefore, the protection scope of the present disclosure shall be subject to the attached claims.

What is claimed is:

1. A Contrast Enhanced Ultrasound (CEUS) method, comprising:
acquiring channel data;
adding the channel data with different attribute information, and obtaining CEUS-complex data;
performing beamforming on the channel data, and obtaining complex channel data corresponding to the channel data; and
obtaining a CEUS image according to the CEUS-complex data and the complex channel data.

2. The method of claim 1, wherein each of ultrasound pulse wave groups is corresponding to the channel data with different attribute information, and adding the channel data with different attribute information, and obtaining CEUS-complex data further comprises:
adding the channel data with different attribute information corresponding to each of ultrasound pulse wave groups, obtaining CEUS channel data, performing beamforming on the CEUS channel data, and obtaining the CEUS-complex data.

3. The method of claim 2, wherein the CEUS-complex data are arranged in a time sequence.

4. The method of claim 2, wherein after performing beamforming on the channel data, and obtaining complex channel data corresponding to the channel data, the method further comprises:
classifying the complex channel data according to the attribute information, and obtaining target complex data corresponding to the attribute information; and
obtaining the CEUS image according to the CEUS-complex data and the complex channel data further comprises:
obtaining the CEUS image according to the CEUS-complex data and the target complex data corresponding to the attribute information.

5. The method of claim 4, wherein classifying the complex channel data according to the attribute information further comprises:
classifying the complex channel data with the same attribute information into a group, wherein the target complex data comprises the group of the complex channel data.

6. The method of claim 2, wherein before performing beamforming on the channel data, the method further comprises:
classifying the channel data with the same attribute information into a group;
performing beamforming on the channel data further comprises:
performing beamforming on the group of channel data.

7. The method of claim 4, wherein the target complex data corresponding to the attribute information are arranged in a time sequence.

8. The method of claim 4, wherein classifying the complex channel data according to the attribute information, and obtaining the target complex data corresponding to the attribute information further comprises:
classifying the complex channel data according to the attribute information, and obtaining initial complex data corresponding to the attribute information; and
performing Doppler filtering on the initial complex data corresponding to the attribute information, and obtaining the target complex data corresponding to the attribute information.

9. The method of claim 2, wherein performing beamforming on the channel data, and obtaining complex channel data corresponding to the channel data further comprises:
performing beamforming on the channel data, performing Doppler filtering on the channel data after beamforming, and obtaining the complex channel data corresponding to the channel data.

10. The method of claim 9, wherein after obtaining the complex channel data corresponding to the channel data, the method further comprises:
classifying the complex channel data according to the attribute information, and obtaining target complex data corresponding to the attribute information; and
obtaining the CEUS image according to the CEUS-complex data and the complex channel data further comprises:
obtaining the CEUS image according to the CEUS-complex data and the target complex data corresponding to the attribute information.

11. The method of claim 1, wherein the channel data is corresponding to N ultrasound pulse waves, N ultrasound pulse waves are divided into M ultrasound pulse wave groups, each of the M ultrasound pulse wave groups comprises m ultrasound pulse waves, N, M, and m are positive integer greater than 1 and satisfy the following formula: M=N/m, and at least one of amplitude, phase, frequency, or distribution of activated elements are different in the m ultrasound pulse waves of each of the M ultrasound pulse wave groups.

12. The method of claim 1, wherein adding the channel data with different attribute information further comprises:
filtering out a non-linear fundamental component from the channel data with different attribute information.

13. The method of claim 4, wherein obtaining the CEUS image according to the CEUS-complex data and the target complex data corresponding to the attribute information further comprises:
generating a mask image according to the target complex data corresponding to the attribute information; and
performing updating on pixel values of the CEUS-complex data by the mask image, and obtaining the CEUS image.

14. The method of claim 13, wherein performing updating on pixel values of the CEUS-complex data by the mask image, and obtaining the CEUS image further comprises:
determining a first pixel in the mask image, wherein a pixel value of the first pixel is a preset value; and
setting a pixel value of a second pixel in the CEUS-complex data to the preset value, and obtaining the CEUS image, wherein the second pixel is corresponding to the first pixel.

15. The method of claim 4, wherein obtaining the CEUS image according to the CEUS-complex data and the target complex data corresponding to the attribute information further comprises:
for each third pixel in the CEUS-complex data, adding a pixel value of a fourth pixel in the target complex data corresponding to the attribute information and a pixel value of the third pixel, and obtaining the CEUS image, wherein the third pixel is corresponding to the fourth pixel.

16. The method of claim 1, wherein the attribute information of the channel data comprises at least one of amplitude, phase, frequency, or distribution of activated elements.

17. The method of claim 11, wherein the ultrasound pulse waves in each of the ultrasound pulse wave groups comprises a wide beam, a plane wave, or a divergent wave.

18. A computer device, comprising a memory and a processor, wherein a computer program is stored in the memory, and the processor is configured to execute the computer program to perform the method of claim 1.

19. A computer-readable storage medium, storing a computer program, wherein the computer program is executed by a processor to perform the method of claim 1.

20. A CEUS apparatus, comprising:
means for acquiring channel data;
means for adding the channel data with different attribute information, and obtaining CEUS-complex data;
means for performing beamforming on the channel data, and obtaining complex channel data corresponding to the channel data; and
means for obtaining a CEUS image according to the CEUS-complex data and the complex channel data.
